(19) United States
(12) Patent Application Publication
(10) Pub. No.: US 2026/0174408 A1
(43) Pub. Date: Jun. 25, 2026

(54) X-RAY COMPUTED TOMOGRAPHY APPARATUS, X-RAY COMPUTED TOMOGRAPHY SYSTEM, AND CONTROL METHOD

(71) Applicants: KEIO UNIVERSITY, Tokyo (JP); Canon Medical Systems Corporation, Otawara-shi (JP)

(72) Inventors: Masahiro JINZAKI, Tokyo (JP); Yoshitake YAMADA, Tokyo (JP); Minoru YAMADA, Hino (JP); Yoichi YOKOYAMA, Tokyo (JP); Yuma TAKEI, Otawara (JP)

(73) Assignees: KEIO UNIVERSITY, Tokyo (JP); Canon Medical Systems Corporation, Otawara-shi (JP)

(21) Appl. No.: 19/430,990

(22) Filed: Dec. 23, 2025

(30) Foreign Application Priority Data
Dec. 25, 2024 (JP) 2024-229138

(51) Int. Cl. A61B 6/00; A61B 6/03; A61B 6/46

(52) U.S. Cl. CPC A61B 6/547; A61B 6/035; A61B 6/4447; A61B 6/461

(57) ABSTRACT

According to one embodiment, an X-ray computed tomography apparatus includes a scanner unit, a stand unit, a display, and processing circuitry. The scanner unit has an imaging system. The stand unit has a tilting mechanism for tilting the scanner unit around a tilt axis. The display is configured to display setting information related to a tilt state of the scanner unit with respect to the stand unit. The processing circuitry is configured to control a manner of displaying the setting information based on the tilt state of the scanner unit for use in an imaging mode.

### CROSS-REFERENCE TO RELATED APPLICATIONS

This application is based upon and claims the benefit of priority from Japanese Patent Application No. 2024-229138, filed Dec. 25, 2024, the entire contents of which are incorporated herein by reference.

### FIELD

Embodiments described herein relate generally to an X-ray computed tomography apparatus, an X-ray computed tomography system, and a control method.

### BACKGROUND

A standing X-ray computed tomography (CT) apparatus capable of imaging a subject in a standing position can image a subject not only in a standing or sitting position but also in a supine position lying on a couch by tilting a scanner unit by 90°.

Both imaging in a standing position and imaging in a sitting position are performed when the angle of the scanner unit is 90° (i.e., when the opening of the scanner unit faces a vertical direction). Thus, when a user such as an operator is to set an imaging mode, it is sometimes difficult to know, based on the appearance of the standing X-ray CT apparatus, which imaging mode—an imaging mode in a standing position or an imaging mode in a sitting position—is currently set.

### BRIEF DESCRIPTION OF THE DRAWINGS

FIG. 1 is a diagram showing an example of a configuration of an X-ray CT apparatus according to an embodiment.

FIG. 2 is a conceptual diagram showing a state of a scanner unit in a standing-imaging mode according to the embodiment.

FIG. 3 is a conceptual diagram showing a state of the scanner unit in a sitting-imaging mode according to the embodiment.

FIG. 4 is a conceptual diagram showing a state of the scanner unit in a supine-imaging mode according to the embodiment.

FIG. 5 is a flowchart illustrating a first example of an operation of the X-ray CT apparatus according to the embodiment.

FIG. 6 is a flowchart illustrating a second example of an operation of the X-ray CT apparatus according to the embodiment.

FIG. 7A is a diagram showing an example of displaying setting information in the standing-imaging mode.

FIG. 7B is a diagram showing an example of displaying setting information in the standing-imaging mode.

FIG. 8 is a diagram showing an example of displaying setting information in the sitting-imaging mode.

FIG. 9 is a diagram showing an example of displaying setting information in the supine-imaging mode.

FIG. 10 is a diagram showing an example of displaying error information according to the embodiment.

### DETAILED DESCRIPTION

In general, according to one embodiment, an X-ray computed tomography apparatus includes a scanner unit, a stand unit, a display, and processing circuitry. The scanner unit has an imaging system. The stand unit has a tilting mechanism for tilting the scanner unit around a tilt axis. The display is configured to display setting information related to a tilt state of the scanner unit with respect to the stand unit. The processing circuitry is configured to control a manner of displaying the setting information based on the tilt state of the scanner unit for use in an imaging mode.

An embodiment of an X-ray CT apparatus, an X-ray CT system, and a control method will be described in detail below with reference to the accompanying drawings. In the embodiment(s) described below, elements assigned the same reference symbols are assumed to perform the same operations, and redundant descriptions thereof will be omitted as appropriate. Hereinafter, an embodiment will be described with reference to the accompanying drawings.

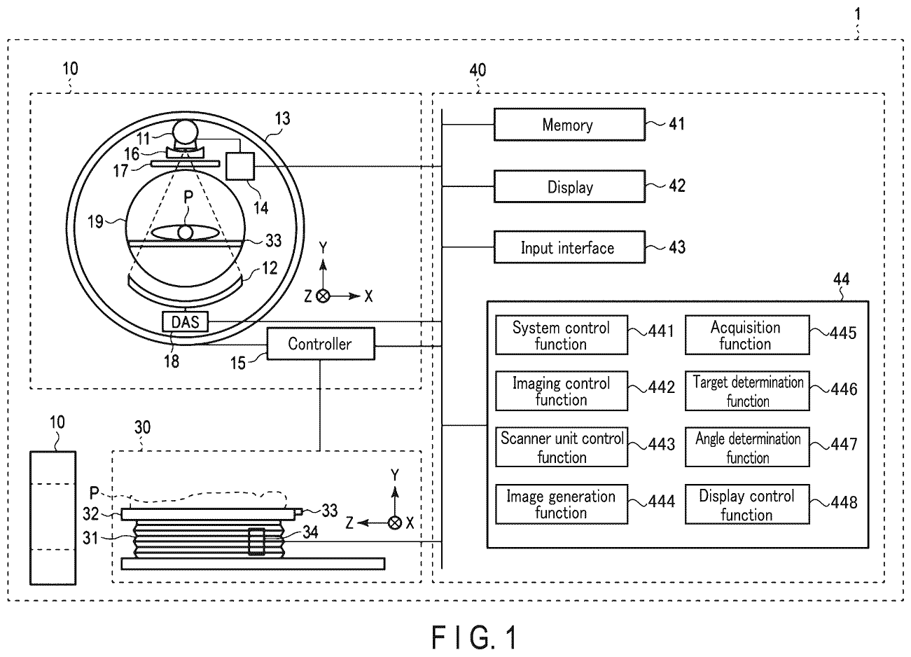

FIG. 1 is a diagram showing an example of a configuration of an X-ray CT apparatus 1 according to an embodiment. As shown in FIG. 1, the X-ray CT apparatus 1 includes a scanner unit 10, a couch 30, and a console 40. FIG. 1 shows a plurality of scanner units 10 for the convenience of explanation; however, the actual number of scanner units 10 may be one or two or more. The scanner unit 10 is a scanner that has a configuration for performing X-ray CT imaging on a subject P. The couch 30 is a carrier device on which the subject P to be subjected to X-ray CT imaging is placed and which regulates the position of the subject P. The console 40 is a computer that controls the scanner unit 10. For example, the scanner unit 10 and the couch 30 are installed in a CT examination room, and the console 40 is installed in a control room adjacent to the CT examination room. The scanner unit 10, the couch 30, and the console 40 are communicably connected to one another wirelessly or by wire. The console 40 need not necessarily be installed in the control room. For example, the console 40 may be installed together with the scanner unit 10 and the couch 30 in the same room. Alternatively, the console 40 may be incorporated into the scanner unit 10.

As shown in FIG. 1, the scanner unit 10 includes an X-ray tube 11, an X-ray detector 12, a rotational frame 13, an X-ray high-voltage device 14, a controller 15, a wedge 16, a collimator 17, and data acquisition circuitry (data acquisition system: DAS) 18.

The X-ray tube 11 emits X-rays to the subject P. Specifically, the X-ray tube 11 includes a cathode that generates thermoelectrons, an anode that generates X-rays by receiving the thermoelectrons travelling from the cathode, and a vacuum tube that holds the cathode and the anode. The X-ray tube 11 is connected to the X-ray high-voltage device 14 via a high voltage cable. The X-ray high-voltage device 14 applies a tube voltage between the cathode and the anode. Thermoelectrons travel from the cathode to the anode upon application of the tube voltage. Tube current flows as thermoelectrons travel from the cathode to the anode. X-rays are generated through collision of the thermal electrons with the anode.

The X-ray detector 12 detects the X-rays that have been emitted from the X-ray tube 11 and have passed through the subject P, and outputs an electric signal corresponding to the detected X-ray dose to the DAS 18. The X-ray detector 12 has a structure in which a plurality of X-ray detection element rows are aligned in a slice direction (row direction), each of the X-ray detection element rows including a plurality of X-ray detection elements aligned in the channel direction. The X-ray detector 12 is, for example, an indirect conversion-type detector including a grid, a scintillator array, and an optical sensor array. The scintillator array includes a plurality of scintillators. The scintillator outputs an amount of light corresponding to an amount of incident X-rays. The grid is arranged on the X-ray incident surface side of the scintillator array, and includes an X-ray shielding plate that absorbs scattered X-rays. The grid may be referred to as a “collimator (one-dimensional collimator or two-dimensional collimator)”. The optical sensor array converts the light to an electric signal corresponding to the amount of light output from the scintillator. For example, a photodiode is used as the optical sensor.

The X-ray detector 12 may be a photon-counting detector.

In the case of a photon-counting detector, the scintillator converts the incident X-rays into photons, the number of which corresponds to the intensity of the incident X-rays. The optical sensor array has the function of amplifying the light received from the scintillator and converting the amplified light into an electric signal, to generate an output signal (energy signal) having a peak value corresponding to the energy of the incident X-rays.

The X-ray detector 12 may be a direct conversion-type detector with a semiconductor element that converts incident X-rays into an electric signal.

The rotational frame 13 is an annular frame that supports the X-ray tube 11 and the X-ray detector 12 rotatably about a rotation axis (Z-axis). Specifically, the rotational frame 13 supports the X-ray tube 11 and the X-ray detector 12 so that the X-ray tube 11 and the X-ray detector 12 face each other. In addition to the X-ray tube 11 and the X-ray detector 12, the rotational frame 13 further supports the X-ray high-voltage device 14 and the DAS 18. The rotational frame 13 is supported by a stationary frame (not shown) so as to be able to rotate about the rotation axis. A rotation mechanism includes, for example, a motor that generates a rotational drive force and a bearing that transmits the rotational drive force to the rotational frame 13 to rotate the rotational frame 13. The motor is provided to the stationary frame, and the bearing is physically connected to the rotational frame 13 and the motor, so that the rotational frame 13 rotates in accordance with the rotational force of the motor. The rotational frame 13 rotates about the rotation axis, whereby the X-ray tube 11 and the X-ray detector 12 are rotated about the rotation axis. The rotational frame 13 is an example of a rotational unit.

The X-ray high-voltage device 14 includes a high-voltage generator and an X-ray controller. The high-voltage generator includes electric circuitry, such as a transformer and a rectifier, and generates high voltage to be applied to the X-ray tube 11 and filament current to be supplied to the X-ray tube 11. The X-ray controller controls output voltage in accordance with the X-rays emitted by the X-ray tube 11. The high-voltage generator may adopt a transformer system or an inverter system. The X-ray high-voltage device 14 may be provided to the rotational frame 13 in the scanner unit 10 or provided to the stationary frame (not shown) in the scanner unit 10.

The wedge 16 adjusts the dose of X-rays emitted to the subject P. Specifically, the wedge 16 attenuates the X-rays so that the dose of X-rays emitted from the X-ray tube 11 to the subject P exhibits a predetermined distribution. For example, a metal plate made of aluminum or the like, such as a wedge filter or a bow-tie filter, is used as the wedge 16.

The collimator 17 limits the range of applying X-rays that have passed through the wedge 16. The collimator 17 slidably supports a plurality of lead plates that shield X-rays and adjusts the shape of slits formed by the lead plates. The collimator 17 may be referred to as an “X-ray diaphragm”.

The DAS 18 reads from the X-ray detector 12 electric signals corresponding to the dose of X-rays detected by the X-ray detector 12. The DAS 18 amplifies the read electric signals and integrates the electric signals during a view period, thereby acquiring detection data with a digital value corresponding to the dose of X-rays during the view period. The detection data is also referred to as projection data. The DAS 18 is implemented by, for example, an application specific integrated circuit (ASIC) equipped with a circuit element capable of generating projection data. The projection data is transmitted to the console 40 via a non-contact data transmitter or the like.

Non-contact or contact-type communication circuitry is provided to each of the rotational frame 13 and the stationary frame, and the communication circuitry enables communication between the units supported by the rotational frame 13 and the stationary frame or an external apparatus of the scanner unit 10. For example, if optical communication is adopted as a non-contact communication method, detection data generated by the DAS 18 is transmitted, via optical communication, from a transmitter with a light-emitting diode (LED), which is provided to the rotational frame 13, to a receiver with a photodiode, which is provided to the stationary frame of the scanner unit 10, and further transferred from the stationary frame to the console 40 by the transmitter. As the communication method, not only the aforementioned communication methods but also a non-contact data transmission method such as a capacitive coupling method and a radio wave method, and a contact-type data transmission method using a slip ring and an electrode brush may be adopted.

The controller 15 controls the X-ray high-voltage device 14 or the DAS 18 to perform X-ray CT imaging in accordance with an imaging control function 442 of processing circuitry 44 of the console 40. The controller 15 includes processing circuitry including a central processing unit (CPU), a micro processing unit (MPU), or the like, and a drive mechanism such as a motor or an actuator. The processing circuitry includes, as hardware resources, a processor such as a CPU or the like and a memory such as a read only memory (ROM), a random access memory (RAM), or the like. The controller 15 implements various functions via a processor executing programs loaded into a memory. Note that the various functions may not be implemented by single processing circuitry. The processing circuitry may be configured by combining a plurality of independent processors, which execute respective programs to implement the respective functions. The controller 15 may be realized by an ASIC or a field programmable gate array (FPGA). The controller 15 may also be realized by a complex programmable logic device (CPLD) or a simple programmable logic device (SPLD).

The controller 15 functions to control the operation of the scanner unit 10 and the couch 30 upon receipt of an input signal from a later-described input interface 43 that is provided to the console 40 or the scanner unit 10 or via a control signal from the processing circuitry 44. For example, the controller 15 performs control to rotate the rotational frame 13, control to tilt the scanner unit 10, and control to operate the couch 30 and the top plate 33 in response to an input signal. The control to tilt the scanner unit 10 can be implemented by the controller 15 rotating the rotational frame 13 about an axis parallel to the X-axis direction based on tilt angle information input through the input interface provided to the scanner unit 10. The controller 15 may be provided to the scanner unit 10 or the console 40. The angle at which the scanner unit 10 is tilted can be set such that, for example, an angle formed by a central line of an opening and a perpendicular line to the floor is in a range of 0°_0 to 90°.

The couch 30 includes a base 31, a support frame 32, the top plate 33, and a couch drive 34. The base 31 is provided on the floor. The base 31 is a housing that supports the support frame 32 movably in a direction perpendicular to the floor (i.e., the Y-axis direction). The support frame 32 is a frame provided on top of the base 31. The support frame 32 supports the top plate 33 slidably along the rotation axis (i.e., the Z-axis). The top plate 33 is a flexible plate on which the subject P is placed.

The couch drive 34 is housed in the housing of the couch 30. The couch drive 34 is a motor or actuator that generates power to move the support frame 32 and the top plate 33 on which the subject P is placed. The couch drive 34 operates in accordance with the control performed by the processing circuitry 44, the console 40, and the like.

The console 40 includes a memory 41, a display 42, an input interface 43, and processing circuitry 44. Data communication between the memory 41, the display 42, the input interface 43, and the processing circuitry 44 is performed via a bus. The console 40 is described as being separate from the scanner unit 10; however, the console 40 or some of the components of the console 40 may be included in the scanner unit 10.

The memory 41 is a storage device, such as a hard disk drive (HDD), a solid-state drive (SSD), or an integrated circuit storage device, which stores various types of information. Other than being an HDD, an SSD, or the like, the memory 41 may be a portable storage medium such as a compact disc (CD), a digital versatile disc (DVD), a Blu-ray (registered trademark) disc (BD), or a flash memory. Alternatively, the memory 41 may be a drive that reads and writes various types of information from and in, for example, a semiconductor memory device such as a flash memory or a RAM. The storage area of the memory 41 may be in the X-ray CT apparatus 1 or in an external storage device connected via a network.

The display 42 displays various types of information. Various types of displays may be discretionarily and suitably adopted as the display 42. For example, a liquid crystal display (LCD), a cathode ray tube (CRT) display, an organic electroluminescence display (OELD), or a plasma display can be used as the display 42. The display 42 may be provided in any place in the control room. The display 42 may be provided to the scanner unit 10. Also, the display 42 may be a desktop-type display, or be configured by a tablet terminal or the like capable of performing wireless communication with the main body of the console 40. One, or two or more projectors may be used as the display 42.

The input interface 43 receives various input operations from an operator, converts the received input operations into electric signals, and outputs the electric signals to the processing circuitry 44. For example, a mouse, a keyboard, a trackball, a switch, a button, a joystick, a touch pad, a touch panel display, or the like can be suitably used as the input interface 43. In the embodiment, the input interface 43 does not necessarily include physical operation components such as a mouse, a keyboard, a trackball, a switch, a button, a joystick, a touch pad, and a touch panel display. Examples of the input interface 43 include processing circuitry for electric signals, which receives an electric signal corresponding to an input operation from an external input device separate from its own apparatus, and outputs this electric signal to the processing circuitry 44. The input interface 43 may be provided to the scanner unit 10. Alternatively, the input interface 43 may be constituted by, for example, a tablet terminal capable of performing wireless communication with the main body of the console 40.

The processing circuitry 44 controls the operation of the entirety of the X-ray CT apparatus 1 in accordance with the electric signal of the input operation output from the input interface 43. The processing circuitry 44 generates image data based on the electric signal output from the X-ray detector 12. For example, the processing circuitry 44 includes a processor, such as a CPU, an MPU, or a GPU, and a memory, such as a ROM or a RAM, as hardware resources. Through a processor that executes a program loaded into the memory, the processing circuitry 44 implements a system control function 441, an imaging control function 442, a scanner unit control function 443, an image generation function 444, an acquisition function 445, a target determination function 446, an angle determination function 447, and a display control function 448.

The respective functions are not necessarily implemented by single processing circuitry. The processing circuitry may be configured by combining a plurality of independent processors, each of which executes respective programs to implement the respective functions.

With the system control function 441, the processing circuitry 44 controls each unit of the X-ray CT apparatus 1 in accordance with the loaded control program. With the system control function 441, the processing circuitry 44 also controls the drive of the couch 30 via the controller 15.

With the imaging control function 442, the processing circuitry 44 controls the X-ray high-voltage device 14, the controller 15, and the DAS 18 in accordance with imaging conditions and performs X-ray CT imaging.

With the scanner unit control function 443, the processing circuitry 44 controls an ascending and descending mechanism and a tilting mechanism so as to move the scanner unit 10 in accordance with an imaging mode. The imaging mode is assumed to be any one of an imaging mode in which the subject P in a standing position is imaged (standing imaging mode), an imaging mode in which the subject P in a sitting position is imaged (sitting imaging mode), or an imaging mode in which the subject P in a supine position is imaged (supine imaging mode).

With the image generation function 444, the processing circuitry 44 subjects projection data related to the subject P to a reconstruction process and generates a CT image. A filter correction backprojection method or a successive approximation reconstruction method is used as the reconstruction process. A reconstruction process that incorporates a denoising process using machine learning into these methods may also be used as the reconstruction process. The processing circuitry 44 converts a CT image to a cross-sectional image of a given cross section or a rendering image in a given direction of a visual point. The conversion is performed based on an input operation received from an operator through the input interface 43. For example, the processing circuitry 44 subjects the reconstructed image data to three-dimensional image processing such as volume rendering, surface volume rendering, pixel value projection processing, multiplanar reconstruction (MPR) processing, curved MPR (CPR) processing, or the like, and generates a rendering image in a given direction of a visual point.

With the acquisition function 445, the processing circuitry 44 obtains at least one of an examination order or an imaging protocol. Also, with the acquisition function 445, the processing circuitry 44 obtains angle information of the scanner unit 10 tilted by the tilting mechanism.

With the target determination function 446, the processing circuitry 44 determines an imaging mode of a subject in the next imaging by referring to at least one of an examination order or an imaging protocol. Also, with the target determination function 446, the processing circuitry 44 determines whether or not the subject P is present in an opening of the scanner unit 10 based on output information of a sensor or a camera. The sensor is, for example, a pressure sensor arranged on a bottom plate. Based on a pressure value, which is output information of a pressure sensor, it may be determined that the subject P is present in the opening of the scanner unit 10 if the pressure value is equal to or greater than a threshold. The camera is, for example, a camera installed on a ceiling or a wall. Based on image information, which is output information of a camera, it may be determined that the subject P is present in the opening of the scanner unit 10 via, for example, an image recognition process.

With the angle determination function 447, the processing circuitry 44 determines whether or not an angle based on the angle information of the scanner unit 10 from the tilting mechanism is an angle that is used in an imaging mode.

With the display control function 448, the processing circuitry 44 causes setting information related to a tilt state of the scanner unit 10 with respect to a later-described stand unit to be displayed on a display unit such as the display 42 based on a tilt state of the scanner unit 10 that is used in an imaging mode implemented for the subject P, and controls a manner of displaying the setting information. Also, with the display control function 448, the processing circuitry 44 causes setting information related to a support tool supporting the subject P used in an imaging mode implemented for the subject P to be displayed on a display unit such as the display 42 based on the support tool, and controls a manner of displaying the setting information. Also, with the display control function 448, the processing circuitry 44 causes a CT image and a rendering image that are generated to be displayed, for example, on the display 42.

The console 40 is described above such that a plurality of functions are performed with a single console; however, a plurality of functions may be performed with separate consoles. The processing circuitry 44 is not limited to a case of being included in the console 40, and may be included in an integrated server that collectively performs processes on projection data obtained by a plurality of medical diagnostic imaging apparatuses. Post-processing may be performed by either the console 40 or an external workstation. The process may also be performed simultaneously by both the console 40 or an external workstation.

The X-ray CT apparatus 1 has various types such as a third-generation CT apparatus and a fourth-generation CT apparatus, any of which is applicable to the present embodiment. The third-generation CT apparatus is of a “rotate/rotate-type,” in which an X-ray tube and a detector integrally rotate about a subject. The fourth-generation CT apparatus is of a “stationary/rotate-type,” in which a number of X-ray detection elements arrayed to form a ring shape are stationary and only an X-ray tube rotates about a subject.

Although not shown, the X-ray CT apparatus 1 may include a communication interface. The communication interface is an interface that connects the X-ray CT apparatus 1 with a workstation, a picture archiving and communication system (PACS), a hospital information system (HIS), a radiology information system (RIS), etc., via a local area network (LAN), etc. The communication interface transmits and receives various kinds of information to and from the connected workstation, PACS, HIS, and RIS.

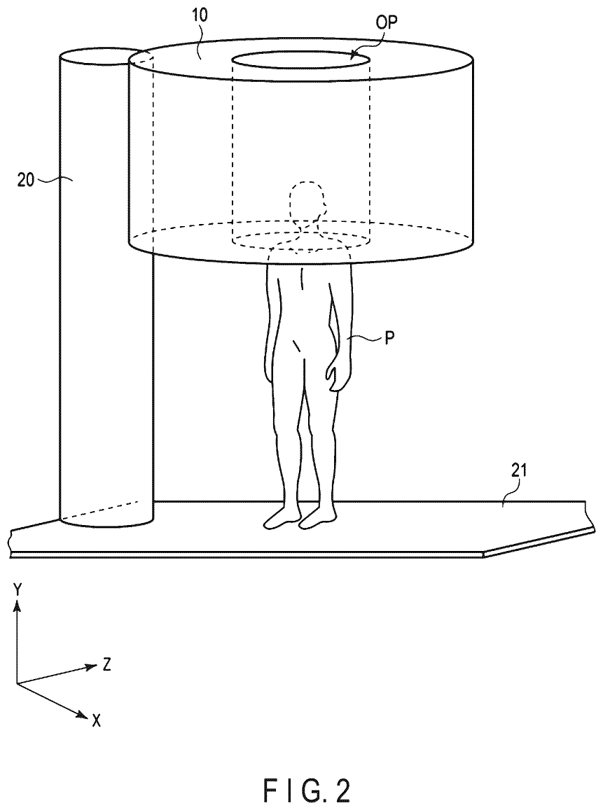

Next, a tilt state of the scanner unit 10 in each imaging mode according to the present embodiment will be described with reference to the conceptual diagrams of FIGS. 2 to 4.

FIG. 2 is a conceptual diagram showing a state of the scanner unit 10 in a standing imaging mode. The present embodiment assumes an X-ray CT apparatus for both imaging in a supine position and imaging in a standing position. That is, in a standing imaging mode, the scanner unit 10 is fixed at a tilt angle where an opening OP faces the vertical direction, and connected to a stand unit 20 erected in the vertical direction. The scanner unit 10 and the stand unit 20 are connected to each other via the tilting mechanism and the ascending and descending mechanism. The tilting mechanism is a mechanism for rotating the scanner unit 10. For example, a general rotating mechanism formed of a gear, a conveyor, and the like may be employed. The ascending and descending mechanism is a mechanism for moving the scanner unit 10 up and down along the vertical direction. For example, a general linear motion mechanism such as a rack and pinion mechanism may be employed. The subject P is inside the opening OP in a standing position and the scanner unit 10 is moved up and down by the ascending and descending mechanism, whereby the subject P is imaged. A pressure sensor or a weight sensor may be embedded in the bottom plate 21 so that the load on the lower side of the scanner unit 10 can be measured.

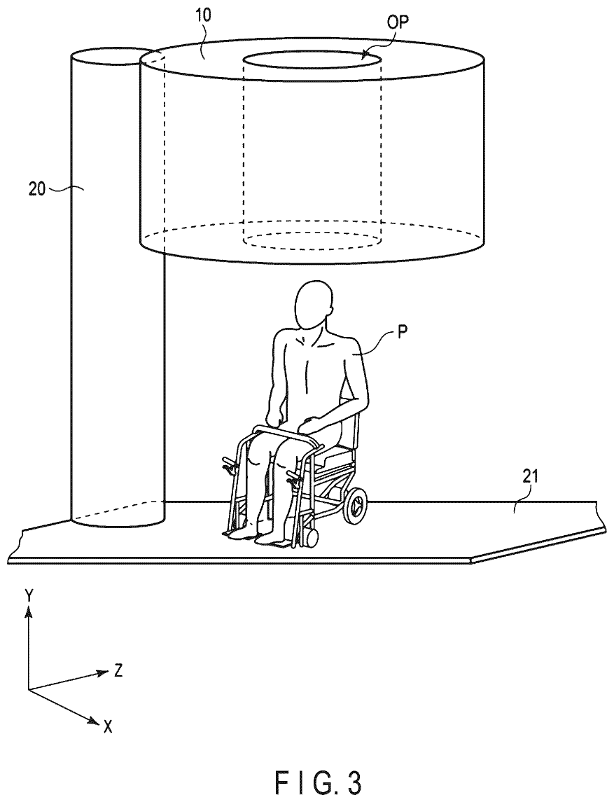

Next, FIG. 3 is a conceptual diagram showing a state of the scanner unit 10 in a sitting imaging mode. In the present embodiment, the sitting imaging mode includes a case where a subject is seated in a chair and a case where a subject is seated in a wheelchair. As in the standing imaging mode, the opening OP of the scanner unit 10 faces the vertical direction. The subject P is inside the opening OP in a state of being seated in a wheelchair or a chair and the scanner unit 10 is moved up and down by the ascending and descending mechanism, whereby the subject P is imaged.

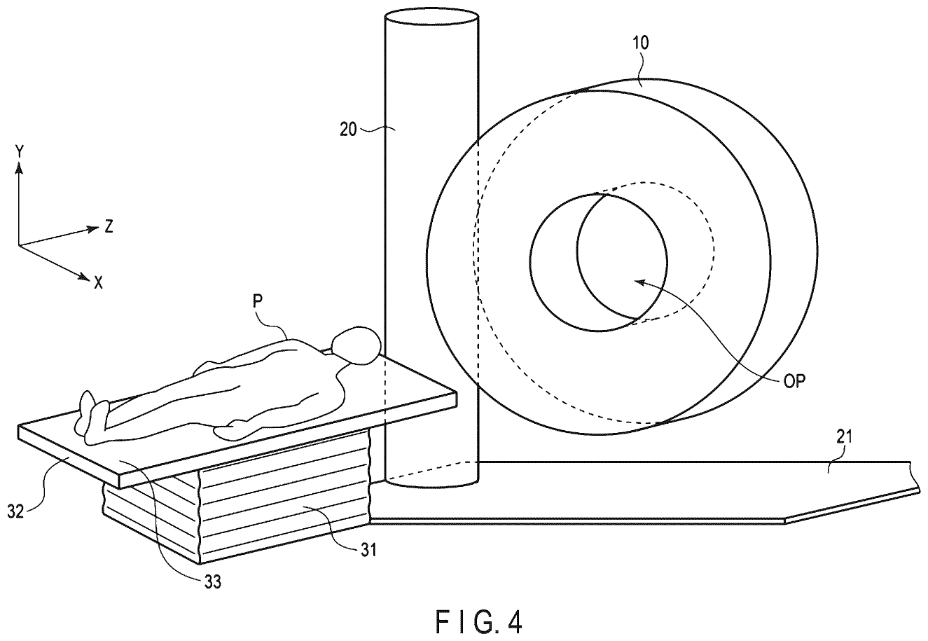

Next, FIG. 4 is a conceptual diagram showing a state of the scanner unit 10 in a supine imaging mode.

In FIG. 4, the scanner unit 10 rotates by 90° from its orientation in the standing imaging mode, and the opening OP faces the horizontal direction. The subject P is in a supine position on the top plate 33 and the top plate 33 is moved to enter the opening OP, whereby the subject P is imaged. That is, the body axis direction of the subject in the standing imaging mode and the body axis direction of the subject in the supine imaging mode are substantially orthogonal to each other.

The scanner unit 10 may be movable in the horizontal direction. For example, the stand unit 20 itself has a drive mechanism for driving the scanner unit 10 along the longitudinal direction of the couch 30 and the scanner unit 10 moves in the horizontal direction together with the stand unit 20. Although the example in which one stand unit 20 is installed is shown, two stand units 20 may be installed with another stand unit 20 installed in an opposing position with the scanner unit 10 interposed therebetween.

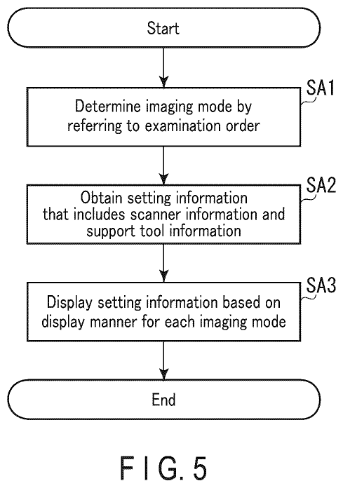

Next, a first example of an operation of the X-ray CT apparatus 1 according to the present embodiment will be described with reference to the flowchart shown in FIG. 5.

In step SA1, with the target determination function 446, the processing circuitry 44 determines which imaging mode is employed by, for example, referring to an examination order.

In step SA2, with the acquisition function 445, by referring to the examination order, the processing circuitry 44 obtains setting information that includes scanner information related to a state, including a position and a tilt angle, of the scanner unit 10 related to the imaging mode and support tool information related to a support tool supporting the subject P.

Since the state of the scanner unit 10 and the support tool necessary for each imaging mode are determined, as in the opening of the scanner unit 10 being made to face the vertical direction and a chair or a wheelchair being provided in the sitting imaging mode, for example, the setting information may be determined in accordance with the imaging mode. Specifically, by, for example, preparing a table of correspondence of the imaging modes with the scanner information and the support tool information, the processing circuitry 44 with the acquisition function 445 may extract the scanner information and the support tool information corresponding to the imaging mode determined in step SA1. Alternatively, the imaging mode and the setting information may be included as the examination order.

In step SA3, with the display control function 448, the processing circuitry 44 causes the setting information to be displayed on, for example, a display based on a display manner predetermined for each imaging mode. The display manner predetermined for each imaging mode refers to a manner in which distinctions are made by, for example, setting blue for a background color in the standing imaging mode, pink for a background color in the sitting imaging mode, and yellow for a background color in the supine imaging mode. The display manner is not limited thereto, and any display manner may be employed as long as it allows a user to know the type of imaging modes at first sight by, for example, changing the display font, changing the display position (layout), or changing the display icon for each imaging mode. Text, that is, textual information, indicating which imaging mode (imaging in a supine position, imaging in a standing position, or imaging in a sitting position) is used can also be displayed on a display. For example, in the supine imaging mode, text such as “imaging in a supine position” or “during imaging in a supine position” is displayed on a display. As a matter of course, the aforementioned display manner and textual information can also be combined as long as a user can easily understand the imaging modes.

Also, with the display control function 448, the processing circuitry 44 highlights the scanner unit 10 and a necessary support tool used in an imaging mode on a schematic diagram based on the scanner information and the support tool information included in the setting information. Examples of the highlighting include a thick line, blinking, coloring, and the like; however, the highlighting is not limited thereto. A method that allows a user to perceive that information is highlighted may be employed. Also, with the display control function 448, the processing circuitry 44 may highlight a direction in which the scanner unit 10 can be tilted and a direction in which the scanner unit 10 can be moved. If, on the other hand, the couch 30, a support tool, and the like are not needed in the imaging mode, a display method may be adopted that makes a display less noticeable compared to highlighted display and normal display prior to highlighted display, such as displaying in a light color, graying out, increasing transmission, or displaying with a broken line. Further, if the couch 30, a support tool, and the like are not used in the imaging mode, non-display may be employed. The “display method that makes display less noticeable” refers to a display method in which display is not highlighted for a user as compared to highlighted display, and can also be rephrased as non-highlighted display including a case of setting non-display. In the case of using highlighted display and non-highlighted display in combination, non-highlighted display may be less noticeable than highlighted display and is preferably less noticeable than normal display. Also, in the case of using normal display and non-highlighted display, non-highlighted display may be less noticeable than normal display and may be non-display.

Thus, highlighting a unit and a support tool necessary in an imaging mode but not highlighting a unit or a support tool unnecessary in the imaging mode allows a user to easily understand the configuration needed in the imaging mode.

With the target determination function 446, the processing circuitry 44 determines an imaging mode implemented in the next imaging by referring to at least one of an examination order or an imaging protocol. With the display control function 448, based on the examination order, the processing circuitry 44 may cause setting information related to the next imaging mode to be displayed after the subject P is imaged in the current imaging mode displayed in step SA3. In this case, after imaging is ended in the current imaging mode, the support tool and the couch 30 may be set to non-highlighted display, and a support tool and the couch 30 used in the next imaging mode may be highlighted.

Furthermore, with the display control function 448, the processing circuitry 44 may cause a message to a user regarding a support tool necessary in the next imaging mode and, in contrast, a support tool not necessary in the next imaging mode to be displayed. For example, in the case of switching from the standing imaging mode to the sitting imaging mode, a message “A chair should be provided.” may be displayed since, for example, a chair is needed. Also, in the case of switching from the sitting imaging mode that uses a chair to the supine imaging mode, a message “The chair should be removed.” may be displayed since the chair is not needed in the supine imaging mode.

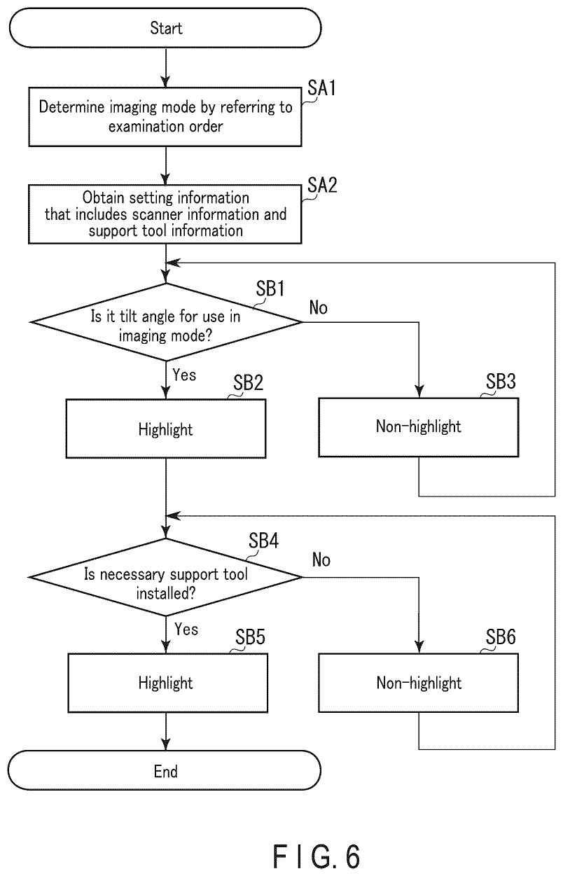

Next, a second example of an operation of the X-ray CT apparatus 1 according to the present embodiment will be described with reference to the flowchart shown in FIG. 6. The second example of an operation differs from the first example of an operation in that the state of the scanner unit 10 and the installation situation of a support tool are grasped and displayed in real time.

Step SA1 and step SA2 are the same as those described in FIG. 5.

In step SB1, with the angle determination function 447, based on the scanner information, the processing circuitry 44 determines whether or not the tilt angle of the scanner unit 10 is an angle that is used in the imaging mode determined in step SA1. For example, if a tilt angle of 15° is generated in the standing imaging mode despite the fact that the tilt angle is set to zero in the examination order, it may be determined that the tilt angle is not an angle that is used in the standing imaging mode. If the tilt angle is an angle that is used in an imaging mode, the process proceeds to step SB2, and if the tilt angle is an angle that is not used in an imaging mode, the process proceeds to step SB3.

In step SB2, with the display control function 448, the processing circuitry 44 highlights the scanner unit 10 when the setting information is displayed based on a display manner predetermined for each imaging mode. For example, a contour may be displayed with a thick line in a schematic diagram of an X-ray CT apparatus and a support tool.

In step SB3, with the display control function 448, the processing circuitry 44 sets the scanner unit 10 to non-highlighted display as described above since imaging is not possible. Thereafter, the process returns to step SB1, and the process is repeated until setting is completed such that imaging in an imaging mode is possible.

In step SB4, with the target determination function 446, the processing circuitry 44 determines whether or not a support tool necessary in the imaging mode determined is installed. Whether or not there is a support tool near the opening of the scanner unit 10 may be determined by, for example, subjecting an image captured by a camera installed in an examination room to an image analysis. By installing a pressure sensor and if a value of the pressure sensor is equal to or greater than a threshold and equal to or less than an average human body weight, it may be determined that there is a support tool. Specifically, in the standing imaging mode, whether or not a pole assisting the standing position of the subject P is vertically installed in the opening of the scanner unit 10 may be determined, and in the sitting imaging mode, whether or not a chair is installed on a lower side of the opening of the scanner unit 10 may be determined.

If a necessary support tool is installed, the process proceeds to step SB5, and if a necessary support tool is not installed, the process proceeds to step SB6.

In step SB5, with the display control function 448, the processing circuitry 44 highlights the installed support tool when the setting information is displayed based on a display manner predetermined for each imaging mode. For example, a contour may be displayed with a thick line in a schematic diagram of an X-ray CT apparatus and a support tool.

In step SB6, with the display control function 448, the processing circuitry 44 sets the installed support tool to non-highlighted display as described above since imaging is not possible. Thereafter, the process returns to step SB4, and the process is repeated until setting is completed such that imaging in an imaging mode is possible.

For convenience of explanation, the setting of the angle of the scanner unit 10 and the setting of the support tool are explained as separate process blocks; however, they may be performed in real time at the same timing. That is, the process from step SB1 to step SB3 and the process from step SB4 to step SB6 may be performed in parallel.

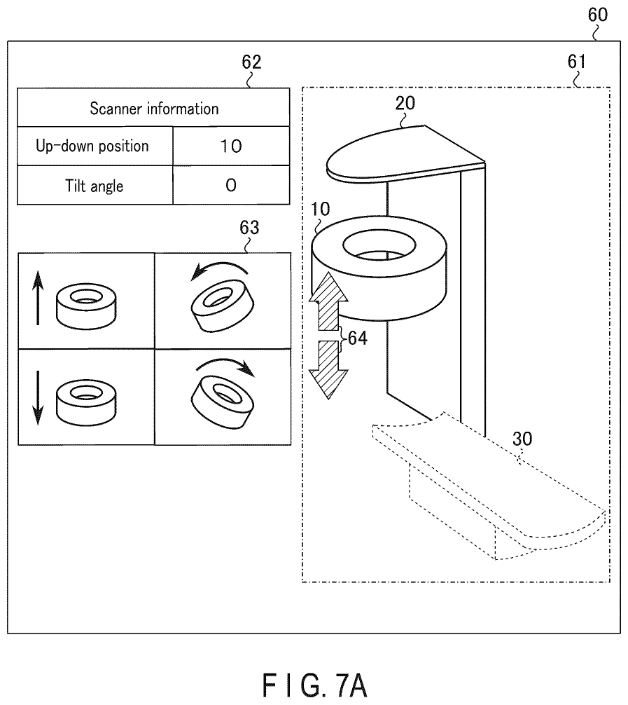

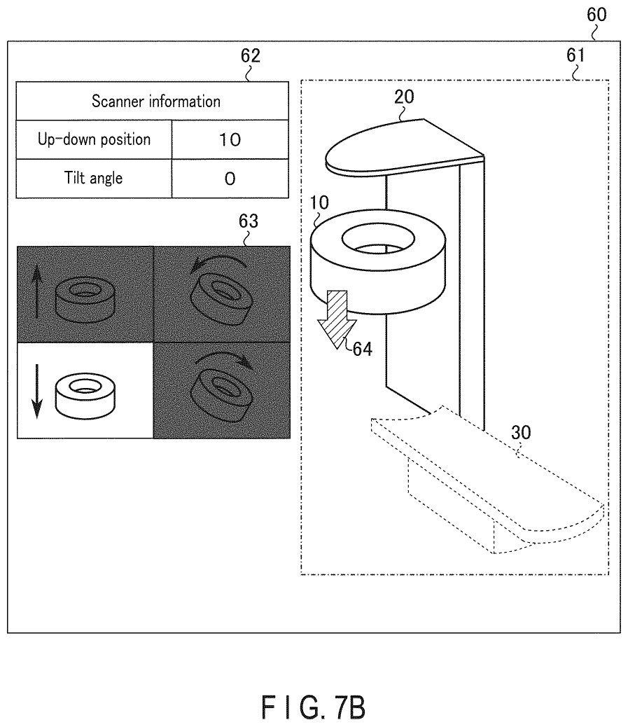

Next, an example of displaying the setting information in the standing imaging mode via the display control function 448 will be described with reference to FIGS. 7A and 7B.

The display screen 60 in FIGS. 7A and 7B shows a schematic diagram region 61 showing the X-ray CT apparatus 1, a scanner information region 62, and an operation panel region 63. It is assumed that the scanner unit 10, the stand unit 20, and the couch 30 are displayed on the schematic diagram region 61, and it is also assumed that support tool information is shown on the schematic diagram region 61. For example, in the standing imaging mode, it is also assumed that a pole for assisting the standing position of the subject P extends in the vertical direction from the bottom plate into the opening. Thus, if a pole is needed, a pole may be shown on the schematic diagram region 61 as support tool information.

The scanner information region 62 displays the position and a value of the tilt angle of the scanner unit 10. The operation panel region 63 is a panel showing a moving direction of the stand unit 20 if the scanner unit 10, the couch 30, and the stand unit 20 are movable. For example, the upward and downward movement and the tilt of the scanner unit 10 and the moving direction of the couch 30 are displayed on the panel. When the panel is selected by a user, the system control function 441 and the scanner unit control function 443 may perform control so as to move the scanner unit 10, the stand unit 20, and the couch 30 that are selected.

As shown in FIG. 7A, with the display control function 448, the processing circuitry 44 may highlight a configuration necessary in the standing imaging mode and display a configuration unnecessary in the standing imaging mode faintly based on the examination order, etc. For example, the scanner unit 10 with its opening facing the vertical direction and the stand unit 20 may be displayed prominently with a thick line, and the couch 30, which is not used in the standing imaging mode, may be set to non-highlighted display as in faint display with a thin line, a broken line, or the like. Alternatively, the couch 30, which is not used in the standing imaging mode, may be non-displayed. This allows a user to easily understand the configuration necessary in the standing imaging mode.

As shown in FIG. 7B, with the display control function 448, the processing circuitry 44 may also cause only a direction in which the scanner unit 10, the stand unit 20, and the couch 30 are movable to be displayed by referring to scanner information. For example, in FIG. 7B, arrows 64 indicating upward and downward are displayed in order to show a case where the scanner unit 10 is movable upward and downward in the vertical direction. Also, if the scanner unit 10 is already moved to the uppermost part within the movable range in the vertical direction, only the arrow 64 facing vertically downward may be displayed. At this time, the processing circuitry 44 with the display control function 448 may cause the operation panel region 63 corresponding to the movable-range direction to be displayed in a highlighted manner, and may cause the operation panel region 63 corresponding to the unmovable-range direction to be displayed in a non-highlighted manner, that is, gray it out or display it faintly, or not even display it. Specifically, if the scanner unit 10 is movable only vertically downward, an operation panel showing a vertically upward movement may be grayed out.

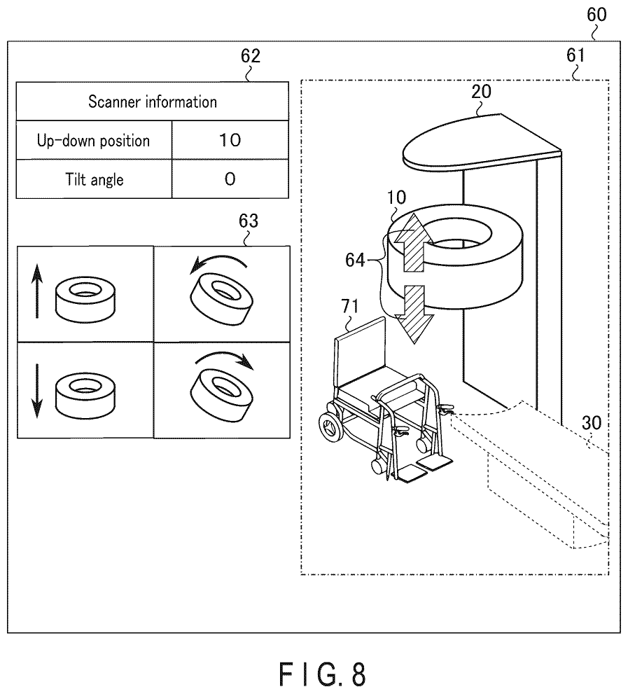

Next, an example of displaying the setting information in the sitting imaging mode via the display control function 448 will be described with reference to FIG. 8.

In FIG. 8, the display screen 60 shows the schematic diagram region 61, the scanner information region 62, and the operation panel region 63, as in FIGS. 7A and 7B. The information displayed in the scanner information region 62 and the operation panel region 63 and the control method are the same as those in the standing imaging mode.

With the display control function 448, the processing circuitry 44 may cause a configuration necessary in the sitting imaging mode to be displayed in a highlighted manner and cause a configuration unnecessary in the sitting imaging mode to be displayed in a non-highlighted manner based on the examination order, etc. For example, the scanner unit 10 with its opening facing the vertical direction and the stand unit 20, and, herein, a wheelchair 71 as support tool information that is used in the sitting imaging mode may be displayed prominently with a thick line, and the couch 30, which is not used in the sitting imaging mode, may be displayed faintly with a thin line, a broken line, or the like. Alternatively, the couch 30, which is not used in the sitting imaging mode, may be non-displayed. This allows a user to easily understand the configuration necessary in the sitting imaging mode. In particular, while the orientation of the opening of the scanner unit 10 is the same as that in the standing imaging mode, displaying the wheelchair 71 allows a user to easily understand that the sitting imaging mode is employed.

Also, with the display control function 448, the processing circuitry 44 may gray out the wheelchair 71 or display the wheelchair 71 with a broken line if the wheelchair 71 is not arranged on the lower side of the scanner unit 10, and highlight the wheelchair 71 with a solid line if the wheelchair 71 is arranged on the lower side of the scanner unit 10, by, for example, analyzing the image captured by a camera. This allows a user to understand that a support tool such as a wheelchair or a chair is needed in the sitting imaging mode and to understand that no support tool has been provided yet if the display is grayed-out or displayed with a broken line.

As in the standing imaging mode, the processing circuitry 44 with the display control function 448 may cause only a direction in which the scanner unit 10 and the stand unit 20 are movable to be displayed by referring to scanner information.

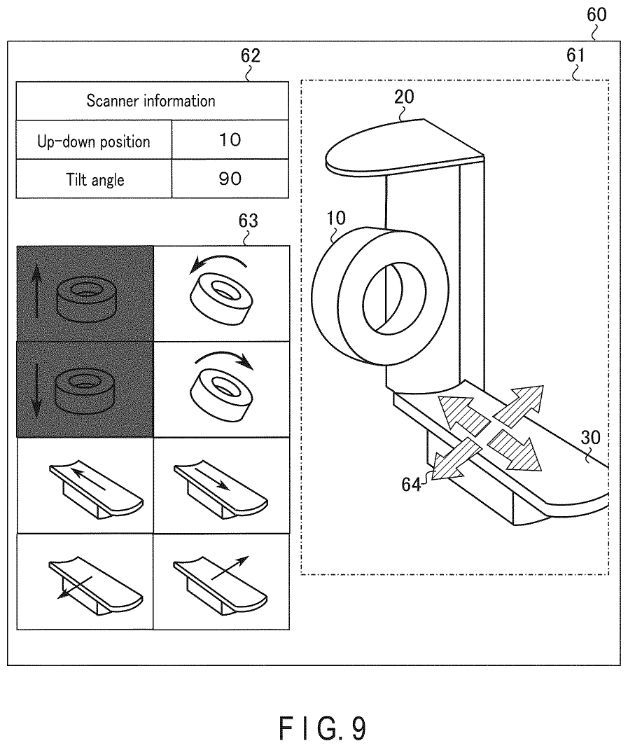

Next, an example of displaying the setting information in the supine imaging mode via the display control function 448 will be described with reference to FIG. 9.

In FIG. 9, the display screen 60 shows the schematic diagram region 61, the scanner information region 62, and the operation panel region 63, as in the standing imaging mode and the sitting imaging mode. The information displayed in the scanner information region 62 and the operation panel region 63 and the control method are the same as those in the standing imaging mode except that information of the couch 30 is added.

With the display control function 448, the processing circuitry 44 may cause a configuration necessary in the supine imaging mode to be displayed in a highlighted manner and cause a configuration unnecessary in the supine imaging mode to be displayed faintly based on the examination order, etc. In the supine imaging mode, the scanner unit 10 with its opening facing the horizontal direction, the stand unit 20, and the couch 30 may be displayed prominently with a thick line. With the display control function 448, the processing circuitry 44 may cause only a direction in which the scanner unit 10, the stand unit 20, and the couch 30 are movable to be displayed by referring to scanner information.

When switching from the standing imaging mode and the sitting imaging mode to the supine imaging mode or vice versa, the scanner unit 10 needs to be tilted by 90°. A case is also assumed where, depending on a user's manual operation situation, the drive of the scanner unit may be terminated without the opening being tilted to the vertical direction or horizontal direction and with a tilt angle remaining present. Thus, with the angle determination function 447, the processing circuitry 44 determines whether or not an angle is an angle that is used in an imaging mode based on the angle information of the scanner unit 10. With the display control function 448, the processing circuitry 44 may cause error information indicating that the scanner unit 10 is not set to a predetermined angle to be displayed if it is determined that an angle based on the angle information is not an angle used in an imaging mode.

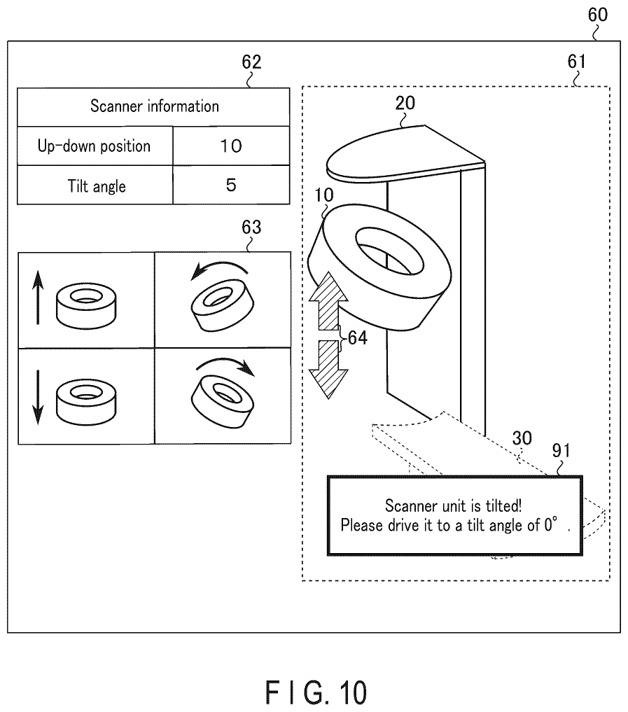

An example of displaying the error information according to the present embodiment will be described with reference to FIG. 10.

With the display control function 448, the processing circuitry 44 causes error information 91 to be displayed on the display screen 60. The error information may be any information, provided that it allows a user to grasp the state of the scanner unit 10 and the installation situation of a support tool, such as error text, an explanation that the scanner unit 10 is not set to a predetermined angle, information related to a state in which the scanner unit 10 is tilted at an angle other than an angle used in a determined imaging mode, and the like.

FIG. 10 assumes a case where imaging is performed in the standing imaging mode, and assumes a case where the opening of the scanner unit 10 turns up to 90° in the vertical direction and the tilt angle is, for example, 5°, not 0°.

With the display control function 448, the processing circuitry 44 may cause a message indicating the error information 91 “The scanner unit is tilted! Please drive it to a tilt angle of 0 °.” to be displayed. Also, a schematic diagram of the scanner unit 10 in a state different from the angle of the scanner unit 10 used in an imaging mode may be displayed in the schematic diagram region 61. That is, the scanner unit 10 may be emphasized such that it has a magnified angle as compared to its actual tilt angle. For example, the scanner unit 10 may be displayed at a tilt angle of 45° while its actual tilt angle is 5°. In this manner, a schematic diagram of the scanner unit 10 in a state of having a tilt angle larger than the actual tilt angle indicated by the angle information may be displayed. This allows a user to easily understand that an orientation of the opening of the scanner unit 10 necessary in an imaging mode is not provided.

In any of the supine imaging mode, the standing imaging mode, and the sitting imaging mode, the processing circuitry 44 with the display control function 448 may notify a user that the tilt angle of the scanner unit 10 is not deviated but set correctly if the tilt angle based on the angle information is the tilt angle of the scanner unit 10 used in an imaging mode. For example, with the display control function 448, the processing circuitry 44 may show the schematic diagram region 61 in a unique background color. Alternatively, a user may be notified, for example, via display of text indicating “○○ imaging mode No tilt angle” or via voice. This makes it possible to explicitly tell a user that the angle of the scanner unit 10 in an imaging mode is correct.

In the examples described above, the display 42 and the processing circuitry 44 are installed in the console 40 of the X-ray CT apparatus; however, the embodiment is not limited thereto. A display device such as the display according to the present embodiment and the processing circuitry 44 may be installed in a portable device such as a smartphone, a tablet terminal, or a laptop PC so that an X-ray CT system configured to be able to remotely communicate with the X-ray CT apparatus 1 that includes the scanner unit 10 and the stand unit 20 implements the process of the present embodiment. Alternatively, a display unit and the processing circuitry 44 including the display control function 448 may be configured separately such that, for example, setting information is displayed on a hanging monitor in an examination room and information can be operated with a tablet terminal at hand.

According to the present embodiment described above, for the scanner unit supported by the stand having the tilting mechanism configured to tilt the scanner unit about a tilting axis, the processing circuitry with the display control function controls a manner of displaying setting information related to a tilt state of the scanner unit and an support tool based on the tilt state of the scanner unit and the support tool supporting a subject that are used in an imaging mode implemented for a subject. In particular, since units and support tools that are needed differ between the standing imaging mode, the sitting imaging mode, and the supine imaging mode, information related to an support tool and a state of the scanner unit related to a position and a tilt angle that are needed for each imaging mode is highlighted whereas units and support tools that are not needed in any one of the imaging modes is set to a non-highlighted manner.

Thus, a user can easily understand a configuration necessary in an imaging mode. That is, a user can easily understand the setting of an imaging mode or the state of the apparatus, and improve the workflow.

The term “processor” used in the above description means, for example, a CPU, a GPU, or circuitry such as an application specific integrated circuit (ASIC), a programmable logic device (e.g., a simple programmable logic device (SPLD), a complex programmable logic device (CPLD), or a field programmable gate array (FPGA)). The processor implements a function by reading and executing a program stored in storage circuitry. The program may be directly incorporated into the circuit of the processor instead of being stored in the storage circuit. In this case, the processor implements the function by reading and executing the program incorporated into the circuit. The function corresponding to the program may be realized by a combination of logic circuits, not by executing the program. Each processor of the present embodiment is not necessarily configured as single circuitry, but may include a plurality of units of independent circuitry to implement the functions of the processor. Furthermore, multiple components may be integrated into a single processor to implement the functions of the processor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography apparatus comprising:
a scanner unit having an imaging system;
a stand unit having a tilting mechanism for tilting the scanner unit around a tilt axis;
a display configured to display setting information related to a tilt state of the scanner unit with respect to the stand unit; and
processing circuitry configured to control a manner of displaying the setting information based on the tilt state of the scanner unit for use in an imaging mode.

2. The X-ray computed tomography apparatus according to claim 1, wherein
the setting information displayed by the display unit further includes information related to a support tool for supporting a subject, and
the processing circuitry is further configured to control the manner of displaying the setting information based on the support tool for use in the imaging mode implemented for the subject.

3. The X-ray computed tomography apparatus according to claim 1, wherein the processing circuitry is configured to highlight the tilt state of the scanner unit in accordance with a type of the imaging mode.

4. The X-ray computed tomography apparatus according to claim 1, wherein the processing circuitry is configured to highlight a direction in which the scanner unit is tiltable or a direction in which the scanner unit is movable.

5. The X-ray computed tomography apparatus according to claim 1, wherein the processing circuitry is configured to change a background color of the setting information in accordance with the imaging mode.

6. The X-ray computed tomography apparatus according to claim 1, wherein the imaging mode is any one of a standing imaging mode, a sitting imaging mode, or a supine imaging mode.

7. The X-ray computed tomography apparatus according to claim 1, wherein the processing circuitry is further configured to:
determine an imaging mode implemented in next imaging by referring to at least one of an examination order or an imaging protocol, and
display the setting information of the imaging mode implemented in the determined next imaging after current imaging is ended.

8. The X-ray computed tomography apparatus according to claim 1, wherein the processing circuitry is further configured to:
obtain angle information related to a tilt angle of the scanner unit;
determine whether or not the tilt angle based on the angle information is a tilt angle for use in the imaging mode; and
display error information related to the scanner unit if it is determined that the tilt angle is not a tilt angle for use in the imaging mode.

9. The X-ray computed tomography apparatus according to claim 8, wherein the processing circuitry is configured to display, as the error information, a schematic diagram of the scanner unit in a state of having a tilt angle larger than an actual tilt angle indicated by the angle information.

10. The X-ray computed tomography apparatus according to claim 1, wherein the processing circuitry is further configured to:
obtain angle information related to a tilt angle of the scanner unit;
determine whether or not the tilt angle based on the angle information is a tilt angle for use in the imaging mode; and
notify a user that the tilt angle of the scanner unit is set correctly if the tilt angle based on the angle information is a tilt angle for use in the imaging mode.

11. The X-ray computed tomography apparatus according to claim 1, wherein the processing circuitry is further configured to determine whether or not a subject is in the scanner unit based on an output from a pressure sensor or a camera.

12. The X-ray computed tomography apparatus according to claim 11, wherein the processing circuitry is further configured to highlight a schematic diagram of the subject in the scanner unit if it is determined that the subject is in the scanner unit.

13. The X-ray computed tomography apparatus according to claim 11, wherein the processing circuitry is further configured to gray out, display with a broken line, or delete the schematic diagram of the subject if it is determined that the subject is not in the scanner unit.

14. An X-ray computed tomography system comprising:
an X-ray computed tomography apparatus including a scanner unit having an imaging system and a stand unit having a tilting mechanism for tilting the scanner unit around a tilt axis;
a display configured to display setting information related to a tilt state of the scanner unit with respect to the stand unit; and
processing circuitry configured to control a manner of displaying the setting information based on the tilt state of the scanner unit for use in an imaging mode.

15. A control method of an X-ray computed tomography apparatus including a scanner unit having an imaging system and a stand unit having a tilting mechanism for tilting the scanner unit around a tilt axis, the method comprising
controlling a manner of displaying setting information related to a tilt state of the scanner unit based on a tilt state of the scanner unit with respect to the stand unit, the tilt state of the scanner unit being for use in an imaging mode.
